(19) United States
(12) Patent Application Publication
(10) Pub. No.: US 2010/0192959 A1
(43) Pub. Date: Aug. 5, 2010

(54) SHAPE MEMORY POLYMER-BASED TRANSCERVICAL DEVICE FOR PERMANENT OR TEMPORARY STERILIZATION

(75) Inventors: Robin Shandas, Boulder, CO (US); Christopher M. Yakacki, Atlanta, GA (US); Devatha P. Nair, Lakewood, CO (US); Kenneth Gall, Atlanta, GA (US); Michael Lyons, Boulder, CO (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: The Regents of the University of Colorado, a body corporate, Denver, CO (US)

(21) Appl. No.: 12/520,399

(22) PCT Filed: Dec. 19, 2007
(86) PCT No.: PCT/US2007/088203

§ 371 Date: Apr. 20, 2010

Related U.S. Application Data
(60) Provisional application No. 60/870,760, filed on Dec. 19, 2006.

(51) Int. Cl. A61F 6/20

(52) U.S. Cl. 128/831

(57) ABSTRACT

Transcervical contraceptive devices (TCDs) are disclosed. The TCDs are constructed of shape memory polymer (SMP) materials capable of assuming a memory shape at physiological temperatures. These SMPTCDs (410) have a post-implantation memory shape that is substantially identical to or slightly larger than the insertion site (420) to adapt to changes that may occur in a fallopian tube. The SMPTCDs (410) may be formed as occlusion devices (i.e., plugs) having a number of different structural features. The SMPTCDs (410) may provide for a temporary or permanent means of contraception.

### CROSS REFERENCE TO RELATED APPLICATIONS

This application claims priority pursuant to 35 U.S.C. §119(e) to U.S. provisional application No. 60/870,760 filed 19 Dec. 2006 and entitled “Shape memory polymer-based transcervical device for permanent or temporary sterilization,” which is hereby incorporated herein by reference in its entirety.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant Nos. EB004481-01A1 and HL067393 awarded by the National Institute of Health.

### BACKGROUND

Family planning and the prevention of pregnancy are very important issues for millions of people worldwide. One type of contraceptive device used by women is the IntraUterine Device (IUD) or the TransCervical Device (TCD). IUDs and TCDs can provide a permanent and effective method of contraception.

There are a number of different types of IUDs and TCDs currently available on the market today. However, there are a number of concerns associated with these currently available TCDs. These concerns include a complicated and painful placement procedure due to the method of deployment used which requires a speculum to widen the cervix, followed by the use of a catheter or tube to deploy the TCD. Additionally, a major reason for the failure of current metal-based TCDs is expulsion due to an improper fit. Furthermore, some of the currently available TCDs may not be effective immediately upon implantation. Some devices require up to three months to become effective after placement. Additionally, some of these IUDs are permanent nonreversible methods of contraception. Thus, there exists a need for a safe, convenient, temporary contraception or sterilization TCD to prevent conception or pregnancy.

Men and women often want to prevent or delay conception or pregnancy, for a number of different reasons. According to the Center for Disease Control and Prevention (CDC), the most common form of birth control in the United States according to a 2002 survey was permanent birth control (including vasectomy and tubal ligation) at 36%. It is estimated that 700,000 tubal ligation procedures and approximately 400,000 vasectomy procedures are performed each year in the United States. Also, the market for this technology encompasses not only women who want permanent birth control, but potentially would extend to women who are using temporary birth control methods because non-incisional alternatives to tubal ligation have not been available until now. Therefore, the future market for TCDs has the potential for significant growth as more people opt for this procedure instead of the other currently available permanent sterilization procedures.

### Shape Memory Polymer (SMP) Materials

An initial paper describing the basic thermomechanical properties of exemplary SMPs was published in 2005 (K. Gall, C. Yakacki, Y. Liu, R. Shandas, and K. S. Anseth. “Thermomechanics of the Shape Memory Effect in Polymers for Biomedical Applications” Journal of Biomedical Materials Research A., 2005; 73A:339-348). Several conference proceedings documenting the thermomechanical properties have also been published (Optimized Thermomechanics of a Shape Memory Polymer Stent to Recover at Body Temperature: ASME Summer Bioengineering, Vail, Colo., 2005; Optimizing the Thermomechanics of Shape Memory Polymers for Biomedical Applications: Mechanics of Materials (McMat), Baton Rouge, La., 2005; Thermomechanics and Manufacturing of Shape Memory Polymers for Biomedical Applications: Graduate Engineering Annual Research Symposium (GEARS), University of Colorado, Boulder, Colo., 2005; Thermomechanics of the Shape Memory Effect in Polymers for Biomedical Applications: Materials Research Society (MRS), Boston, Mass. 2004; Thermomechanics of the Shape Memory Effect in Polymers for Biomedical Applications: Society of Engineering Science (SES), Lincoln, Neb., 2004). Certain devices having SMP materials have been tested in vitro for mechanical properties.

Shape memory materials are defined by their capacity to recover a predetermined shape after significant mechanical deformation (K. Otsuka and C. M. Wayman, “Shape Memory Materials” New York: Cambridge University Press, 1998). The shape memory effect is typically initiated by a change in temperature and has been observed in metals, ceramics, and polymers. From a macroscopic point of view, the shape memory effect in polymers differs from ceramics and metals due to the lower stresses and larger recoverable strains achieved in polymers.

Basic thermomechanical response of SMP materials is defined by four critical temperatures. The glass transition temperature, T_{g}, is typically represented by a transition in modulus-temperature space and can be used as a reference point to normalize temperature. SMPs offer the ability to vary T_{g} over a temperature range of several hundred degrees by control of chemistry or structure. The predeformation temperature, T_{d}, is the temperature at which the polymer is deformed into its temporary shape. Depending on the required stress and strain level, the initial deformation at T_{d} can occur above or below T_{g} (Y. Liu, K. Gall, M. L. Dunn, and P. McCluskey, “Thermomechanical Recovery Couplings of Shape Memory Polymers in Flexure.” Smart Materials & Structures, vol. 12, pp. 947-954, 2003). The storage temperature, Tₛ, represents the temperature in which no shape recovery occurs and is equal to or below T_{d}. At the recovery temperature, Tᵣ, the shape memory effect is activated, which causes the material to recover its original shape, and is typically in the vicinity of T_{g}. Recovery can be accomplished isothermally by heating to a fixed Tᵣ and then holding, or by continued heating up to and past Tᵣ. From a macroscopic viewpoint, a polymer will demonstrate a useful shape memory effect if it possesses a distinct and significant glass transition (B. Sillion, “Shape memory polymers,” Act. Chimique., vol. 3, pp. 182-188, 2002) a modulus-temperature plateau in the rubbery state (C. D. Liu, S. B. Chun, P. T. Mather, L. Zheng, E. H. Haley, and E. B. Coughlin, “Chemically cross-linked polycyclooctene: Synthesis, characterization, and shape memory behavior.” Macromolecules. Vol. 35, no. 27, PP. 9868-9874, 2002) and a large difference between the maximum achievable strain, εₘₐₓ, during deformation and permanent plastic strain after recovery, εₚ (F. Li, R. C. Larock, “New Soybean Oil-Styrene-Divinylbenzene Thermosetting Copolymers. V. Shape memory effect.” J. App. Pol. Sci., vol 84, pp. 1533-1543, 2002). The difference εₘₐₓ−εₚ is defined as the recoverable strain, ε_{recover}, while the recovery ratio is defined as ε_{recover}/εₘₐₓ.

The microscopic mechanism responsible for shape memory in polymers depends on both chemistry and structure (T. Takahashi, N. Hayashi, and S. Hayashi, “Structure and properties of shape memory polyurethane block copolymers.” J. App. Pol. Sci., vol 60, pp. 1061-1069, 1996; J. R. Lin and L. W. Chen, “Study on Shape-Memory Behavior of Polyether-Based Polyurethanes. II. Influence of the Hard-Segment Content.” J. App. Pol. Sci., vol 69, pp. 1563-1574, 1998; J. R. Lin and L. W. Chen, “Study on Shape-Memory Behavior of Polyether-Based Polyurethanes. I. Influence of soft-segment molecular weight.” J. App. Pol. Sci., vol 69, pp. 1575-1586, 1998; F. Li, W. Zhu, X. Zhang, C. Zhao, and M. Xu, “Shape memory effect of ethylene-vinyl acetate copolymers.” J. App. Poly. Sci., vol. 71, pp. 1063-1070, 1999; H. G. Jeon, P. T. Mather, and T. S. Haddad, “Shape memory and nanostructure in poly(norbornyl-POSS) copolymers.” Polym. Int., vol. 49, pp. 453-457, 2000; H. M. Jeong, S. Y. Lee, and B. K. Kim, “Shape memory polyurethane containing amorphous reversible phase.” J. Mat. Sci., vol. 35, pp. 1579-1583, 2000; A. Lendlein, A. M. Schmidt, and R. Langer, “AB-polymer networks based on oligo(epsilon-caprolactone) segments showing shape-memory properties.” Proc. Nat. Acad. Sci., vol. 98, no. 3, pp. 842-847, 2001; G. Zhu, G. Liang, Q. Xu, and Q. Yu, “Shape-memory effects of radiation crosslinked poly(epsilon- caprolactone).” J. App. Poly. Sci., vol. 90, pp. 1589-1595, 2003). The primary driving force for shape recovery in polymers is the low conformational entropy state created and subsequently frozen during the thermomechanical cycle (C. D. Liu, S. B. Chun, P. T. Mather, L. Zheng, E. H. Haley, and E. B. Coughlin, “Chemically cross-linked polycyclooctene: Synthesis, characterization, and shape memory behavior.” Macromolecules. Vol. 35, no. 27, pp. 9868-9874, 2002). If the polymer is deformed into its temporary shape at a temperature below T_{g}, or at a temperature where some of the hard polymer regions are below T_{g}, then internal energy restoring forces will also contribute to shape recovery. In either case, to achieve shape memory properties, the polymer must have some degree of chemical crosslinking to form a “memorable” network or must contain a finite fraction of hard regions serving as physical crosslinks.

### SUMMARY

Transcervical devices constructed of shape memory polymer (SMP) materials are disclosed herein. These SMP devices are capable of assuming a memory shape at physiological temperatures and may be used to provide temporary or permanent sterilization by blocking the fallopian tubes. These SMP devices have a post-implantation memory shape that is substantially identical to the insertion site, or have a unique functional shape, and may adapt to changes in the fallopian tubes as needed.

### BRIEF DESCRIPTION OF THE DRAWINGS

FIGS. 1A-1D are sequential images of deployment of a SMPTCD using a specially designed catheter.

FIG. 2 illustrates an exemplary catheter for deployment of a SMPTCD.

FIG. 3 illustrates an exemplary bulb-shaped activated device held by a pair of tweezers.

FIG. 4 illustrates an exemplary bulb-shaped device with an elongated end deployed within a clear tube.

FIG. 5 illustrates a SMPTCD having multiple bulb-shaped portions.

FIGS. 6A-6C illustrate sequential images of deployment (by a catheter) and expansion of a bulb-shaped SMPTCD plug within a fallopian tube.

FIGS. 7-10 illustrate SMPTCDs having hooks, anchors, or barbs.

FIG. 11 and FIG. 12 illustrate SMPTCDs having a wire or wires formed therein.

FIG. 13 illustrates a partially activated coil-shaped SMPTCD.

FIG. 14 illustrates an uncoiled SMPTCD.

FIG. 15 illustrates an exemplary coiled SMPTCD having swollen hydrogel material fully occluding a clear tube.

FIG. 16 illustrates a cross-sectional view of the unrolling and expansion of a coiled SMPTCD.

FIG. 17 illustrates a cross-sectional view of the swelling of a coiled SMPTCD having a hydrogel material therein.

FIGS. 18A-C illustrate sequential images of deployment (by a catheter) and expansion of a coiled SMPTCD within a clear tube.

FIG. 19 illustrates a SMPTCD having a bulb-shaped portion and an SMP stent.

FIG. 20 illustrates a chemical structures of a hydrogel 2-hydroxyethyl methacrylate (2-HEMA).

### DETAILED DESCRIPTION

Devices for permanent or temporary sterilization or contraception using unique blends of Shape Memory Polymer (SMP) materials are disclosed herein. Also disclosed are methods and materials for manufacturing these devices. Device designs include but are not limited to plugs, coils, and variations of each. These devices may be variously referred to herein as devices, SMP devices, SMP-based devices, SMP-based TCDs, SMP TCDs, or SMPTCDs, but all references are to devices comprising, in various amounts, SMP-based materials for use in sterilization or contraception as TCDs or IUDs.

### Shape Memory Materials

The technology disclosed herein utilizes SMP-based materials, as disclosed in U.S. Provisional Application Ser. No. 60/788,540 entitled Shape Memory Polymer Medical Device and in International PCT Application No. PCT/US2006/060297 entitled A Polymer Formulation A Method of Determining A Polymer Formulation and A Method of Determining a Polymer Fabrication, which are both hereby incorporated by reference for all that they disclose.

SMPs have significant capacity to change shape. SMP materials have the ability to activate with a mechanical force under the application of a stimulus. The stimulus may be light, heat, chemicals, or other types of energy or stimuli. The thermomechanical response of SMP materials can be controlled to predict and optimize shape-memory properties. Polymer systems may be designed and optimized to a high degree of tailorability that are capable of adapting and responding to patients' needs for biomedical applications such as stenting, orthopedic fixation, IUDs, closing wounds, repairing aneurisms, etc.

More than one method may be used to design shape memory polymers for use in medical devices, such as TCDs or IUDs. In one method, the polymer transition temperature is tailored to allow recovery at the body temperature, Tᵣ˜T_{g}˜37° C. (A. Lendlein and R. Langer, “Biodegradable, elastic shape-memory polymers for potential biomedical applications.” Science, vol. 296, pp. 1673-1676, 2002). The distinct advantage of this approach is the utilization of the body's thermal energy to naturally activate the material. The disadvantage of this approach, for some applications, is that the mechanical properties (e.g., stiffness) of the material are strongly dependent on T_{g}, and would be difficult to alter in the device design process. In particular, it would be difficult to design an extremely stiff device when the polymer T_{g} is close to the body temperature due to the compliant nature of the polymer. Another possible disadvantage is that the required storage temperature, Tₛ, of a shape memory polymer with T_{g}˜37° C. will typically be below room temperature requiring “cold” storage prior to deployment. In an alternative method, the recovery temperature is higher than the body temperature Tᵣ˜T_{g}>37° C. (M. F. Metzger, T. S. Wilson, D. Schumann, D. L. Matthews, and D. J. Maitland, “Mechanical properties of mechanical actuator for treating ischemic stroke,” Biomed. Microdevices, vol. 4, no. 2, pp. 89-96, 2002; D. J. Maitland, M. F. Metzger, D. Schumann, A. Lee, T. S. Wilson, “Photothermal properties of shape memory polymer micro-actuators for treating stroke.” Las. Surg. Med., vol. 30, no. 1, pp. 1-11, 2002). The advantage of the second method is that the storage temperature can be equal to room temperature facilitating easy storage of the device and avoiding unwanted deployments prior to use. The main disadvantage of the second method, for some applications, is the need to locally heat the polymer to induce recovery. Local damage to some tissues in the human body commences at temperatures approximately 5 degrees above the body temperature through a variety of mechanisms including apoptosis and protein denaturing. Advocates of the second approach use local heating bursts to minimize exposure to elevated temperatures and circumvent tissue damage. The use of one method over the other is a design decision that depends on the targeted body system and other device design constraints such as required in-vivo mechanical properties.

Any polymer than can recover an original shape from a temporary shape by application of a stimulus such as temperature is considered an SMP. The original shape is set by processing and the temporary shape is set by thermo-mechanical deformation. A SMP has the ability to recover large deformation upon heating. The present TCDs are made from SMP-based materials, which can subsequently be compressed or compacted and inserted into a fallopian tube, and deployed or expanded by an increase in temperature. The ability for the device to be deployed will have the benefit of allowing surgeons to easily install the device, as well as provide an optimal loading configuration.

A polymer is a SMP if the original shape of the polymer is recovered by heating it above a shape recovery temperature, or deformation temperature (T_{d}), even if the original molded shape of the polymer is destroyed mechanically at a lower temperature than T_{d}, or if the memorized shape is recoverable by application of another stimulus. Any polymer that can recover an original shape from a temporary shape by application of a stimulus such as temperature may be considered a SMP. In certain embodiments, the shape may be smooth in texture. In other embodiments, the shape may range from smooth to fully textured. In alternative embodiments, the shape may be partially textured.

A SMP material or network may include dissolving materials which may include part of the network or may be included in the formulation of the network before the network is polymerized (e.g., as an aggregate, mixed into the formulation). Dissolving materials may include materials that disperse over time, even if the material or part of the material does not actually dissolve or enter into a solution with a solvent. In other words, a dissolving material as used herein may be any material that may be broken down by an anticipated external environment of the polymer. In one embodiment, a dissolving material is a drug which elutes out of a SMP network. A dissolving material may be attached by chemical or physical bonds to the polymer network and may become disassociated with the polymer network over time.

Dissolving materials may be used to create surface roughness, for example, in order to increase biocompatibility of the network. In one embodiment, the dissolving material may initially form a part of the surface of the SMP network, and leave behind a rougher SMP surface after the dissolving material has dissolved. In another embodiment, the dissolving material may be placed within the body of the SMP network, and upon dissolving may create an impression in the surface of the SMP by allowing the SMP to collapse due to the dissolution of the dissolving material within the body of the SMP.

Dissolving materials, through their dissolution over time, may be used for many purposes. In one embodiment, the dissolution of a material may affect a dissolution or breaking up of a biomedical device over time. In another embodiment, the dissolution of a material may elute a drug, achieving a pharmacological purpose. Medications or drugs can be infused into the SMPTCDs to aid in contraception. In some embodiments medications or drugs may be coated onto surfaces of the SMPTCDs. SMPTCD design may allow greater amounts of drugs to be infused into the polymer than with current polymer-coated metal TCDs or IUDs.

The matrix of the SMP-based material may be supplemented with a variety of drugs during the polymerization process or post-processing. For example, drugs to be added may include anti-inflammatory, pro-contraceptive, and anti-thrombotic drugs. These drugs can be added by injection into the liquid polymer before UV curing. Drugs may also be added to the SMPTCD post-polymerization using various surface modification techniques such as plasma deposition, for example.

An initial surface of an exemplary SMPTCD may be a rough surface. In one embodiment, an initial rough surface may include a dissolving material. In another embodiment, an initial rough surface may be created by including dissolving material inside a SMP network. Once the material has dissolved, a surface with a different roughness may be left behind. In one embodiment, a smooth surface is left after a dissolving material has dissolved. In another embodiment, a surface rougher than the initial is left behind after a dissolving material has dissolved. In another embodiment, a surface with a different type of roughness is left after a dissolving material has dissolved. For example, an initial surface may have roughness in a random pattern and a surface left after a dissolving material has dissolved may have a roughness that is ordered and repeating.

In certain embodiments, the SMP polymer segments can be natural or synthetic, although synthetic polymers are preferred. The polymer segments may be non-biodegradable. Non-biodegradable polymers used for medical applications preferably do not include aromatic groups, other than those present in naturally occurring amino acids. The SMP utilized in the TCDs disclosed herein may be nonbiodegradable. In some implementations, it may be desirable to use biodegradable polymers in the SMPTCDs, for example, when temporary sterilization is desired.

The polymers are selected based on the desired glass transition temperature(s) (if at least one segment is amorphous) or the melting point(s) (if at least one segment is crystalline), which in turn is based on the desired application, taking into consideration the environment of use. Representative natural polymer blocks or polymers include proteins such as zein, modified zein, casein, gelatin, gluten, serum albumin, and collagen, and polysaccharides such as alginate, celluloses, dextrans, pullulane, and polyhyaluronic acid, as well as chitin, poly(3-hydroxyalkanoate), especially poly(.beta.-hydroxybutyrate), poly(3-hydroxyoctanoate), and poly(3-hydroxyfatty acids). Representative natural biodegradable polymer blocks or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein, and copolymers and blends thereof, alone or in combination with synthetic polymers.

Representative synthetic polymer blocks or polymers include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt. These are collectively referred to herein as “celluloses.”

Representative synthetic degradable polymer segments include polyhydroxy acids, such as polylactides, polyglycolides and copolymers thereof poly(ethylene terephthalate); polyanhydrides, poly(hydroxybutyric acid); poly(hydroxyvaleric acid); poly[lactide-co-(.epsilon.-caprolactone)]; poly[glycolide-co-(.epsilon.-caprolactone)]; polycarbonates, poly(pseudo amino acids); poly(amino acids); poly(hydroxyalkanoate)s; polyanhydrides; polyortho esters; and blends and copolymers thereof. Polymers containing labile bonds, such as polyanhydrides and polyesters, are well known for their hydrolytic reactivity. Their hydrolytic degradation rates can generally be altered by simple changes in the polymer backbone and their sequence structure.

Examples of non-biodegradable synthetic polymer segments include ethylene vinyl acetate, poly(meth)acrylic acid, polyamides, polyethylene, polypropylene, polystyrene, polyvinyl chloride, polyvinylphenol, and copolymers and mixtures thereof. The polymers can be obtained from commercial sources such as Sigma Chemical Co., St. Louis, Mo.; Polysciences, Warrenton, Pa.; Aldrich Chemical Co., Milwaukee, Wis.; Fluka, Ronkonkoma, N.Y.; and BioRad, Richmond, Calif. Alternately, the polymers can be synthesized from monomers obtained from commercial sources, using standard techniques.

### Prototype SMPTCD Designs

Prototype SMPTCDs have been designed and manufactured. A device formed from SMP material is capable of assuming a memory shape at physiological temperatures and may be used in a number of different applications. A device formed from SMP material may have a post-implementation memory shape that is substantially identical to the insertion site. Other devices formed from SMP materials may have a unique functional shape, the ability adapt to uniquely sized vessels, and/or the ability to grow with a vessel as needed. A device formed from SMP material may be compacted to a very small size for minimally invasive insertion into a vessel lumen and may then expand to its memory shape once it is placed internally and subjected to stimuli, such as body heat.

The thermomechanical behavior of the SMP material can be optimized to physiological conditions by controlling the SMP modulus, visco-elastic properties, and damping coefficient (tan delta). These properties can be tailored to allow the diameter of the TCD to increase as the diameter of the fallopian tube increases, thereby allowing the TCD to adapt to changing size as may occur due to muscular action such as peristaltic action, due to the adaptive response of the fallopian tube to the implant, or with patient growth. Further, the TCDs disclosed herein may be formed using any percentage or amount of SMP in combination with other materials, if desired.

The SMP-based TCDs disclosed here have a number of unique characteristics due to the incorporation of the SMP materials. The SMP-based TCD has the ability to be highly compacted for delivery yet may expand to accommodate large, and/or non-standard anatomical geometries. The thermomechanical properties of the SMP material offer a greater level of customizability than standard metal-based and coated TCDs. Customizability includes, for example, the ability to tailor mechanical properties such as rubbery modulus, deployment time, and device conformability. Also, as shown by in vitro proof-of-concept studies, SMP-based TCDs are immediately effective upon implantation. These devices can also provide a permanent, yet reversible, method of contraception. In typical cases, a single SMP-based TCD may be inserted into each of the two fallopian tubes of a woman to prevent pregnancy. In other cases, it may be necessary or desirable to utilize fewer or additional SMP-based TCDs, but this will be determined by a qualified physician.

SMPTCDs have been manufactured having a maximum diameter of between about 2-3 mm (when expanded/activated) and minimum diameters of between about 1.2-1.5 mm (when compacted). Of course, both smaller and larger devices may be manufactured. Successful trials of SMPTCDs in a benchtop model of a fallopian tube were completed using a device which expanded from 1.5 mm to 2.4 mm with complete recovery. Flow visualization studies have shown that the SMP devices provide a liquid-tight seal and thus are immediately effective. They can also be retrieved back into a catheter to create a device that can be retrieved subsequently using catheter approaches. Heating to activate or stimulate the SMP-based material may be accomplished via several methods including external methods such as focused ultrasound or magnetic energy or internal methods wherein a catheter is used to deliver electricity, RF energy, ultrasound, or direct heat to the polymer.

The SMPTCDs may be fabricated from copolymer networks uniquely formulated for the particular requirements of the TCD application including biocompatibility, substantial memory shape, and softer mechanical properties. In one embodiment, the copolymer network consists of two acrylate-based monomers. In one example of this embodiment, tert-butyl acrylate may be crosslinked with poly (ethylene glycol)ₙ dimethacrylate (PEGDMA) via photopolymerization to form a cross-linked network. One subset of this formulation may consist of 10 wt % PEGDMA with a Mₙ=1000 and remainder tert-butyl acrylate with 0.1 wt % photoinitiator (2,2 dimethoxy-2-phenylacetopenone). This formulation has been used with good results for the prototype bulb-shaped device described above. This formulation was selected to match several design needs of the SMPTCDs. The polymer network has a glass transition temperature T_{g}˜45° C., which offers shape memory activation along with a reasonably soft compliance at body temperature. Furthermore, it has a low rubbery modulus of approximately 1-2 MPa, which is indicative of a low degree of crosslinking that allows for greater packaging deformations and higher strains to failure.

The SMP material may be further varied to enhance desired properties. The SMP material may be photopolymerized from several different monomers and/or homopolymers to achieve a range of desired thermomechanical properties. An SMP formed from three or more monomers and/or homopolymers may achieve a range of rubbery modulus to glass transition temperature, rather than a strictly linear relationship between these two thermomechanical properties. For example, tert-butyl acrylate may be substituted by 2-hydroxyethyl methacrylate or methyl methylacrylate to create either more hydrophilic or stronger networks, if desired. Additionally, if a hydrophilic monomer such as 2-hydroxyethyl methacrylate is substituted for tert-butyl acrylate, the SMPTCD has the ability to further swell post-implantation through hydrogel mechanisms. The swelling post-implantation may provide for further expansion of the SMPTCD, which allows the SMPTCD to adapt to changes in fallopian tube size after implantation and keep the SMPTCD in place even if the fallopian tube changes or adjusts in size, shape or curvature.

Because SMP material require both a thermal transition and form of crosslinking to possess shape-memory characteristics, the polymer is typically synthesized from a linear chain building mono-functional monomer (tert-butyl acrylate) and a crosslinking di-functional monomer (poly (ethylene glycol) dimethacrylate). Because the crosslinking monomer has two methacrylate groups, one at each end, it is possible to connect the linear chains together. This linear monomer portion can be used to help control the glass transition temperature of the network as well as its overall tendency to interact with water. Thus, the linear portion of the network remains an important and tailor-able portion of the composition.

In some implementations it may be desirable to use 2-hydroxyethyl methacrylate (2-HEMA) in place of, or in conjunction with, tert-butyl acrylate. 2-hydroxyethyl methacrylate (2-HEMA) is illustrated in FIG. 20. 2-HEMA, also known as ethylene glycol methacrylate, forms a highly hydrophilic polymer and is most known for its use in contact lenses. 2-HEMA has a similar structure to the crosslinking monomer and has a similar glass transition temperature (˜70° C.), as compared to tert-butyl acrylate (˜60° C.). By incorporating 2-HEMA into the polymer synthesis, it is possible to create SMPs with a higher affinity toward water, which will cause the polymer to soften and swell by absorbing water over time. The degree by which the polymer will swell will be controlled by the amount of 2-HEMA and the amount of crosslinking within the matrix.

The SMPTCD can be manufactured by several methods including, for example, injection molding or blow molding. In one exemplary method, a SMPTCD may be manufactured by injecting a liquid monomer for formulation into an appropriate glass mould and photopolymerizing. A Teflon® mould of specific shape and geometric considerations may also be used in certain designs along with the glass tube moulds. Glass tube moulds may be blown to match specific geometrical parameters and allow for the SMPTCDs to be patient size- and shape-specific if needed. Once the polymer is cured, the glass mould may be gently broken and the SMPTCD may be removed. The device may then be uncoiled or compacted through an extrusion die at room temperature and cooled in a freezer to lock in this temporary packaged state. The device is then removed from the die and placed in a catheter for implantation.

Placement and/or Retrieval of SMPTCDs Via Catheter

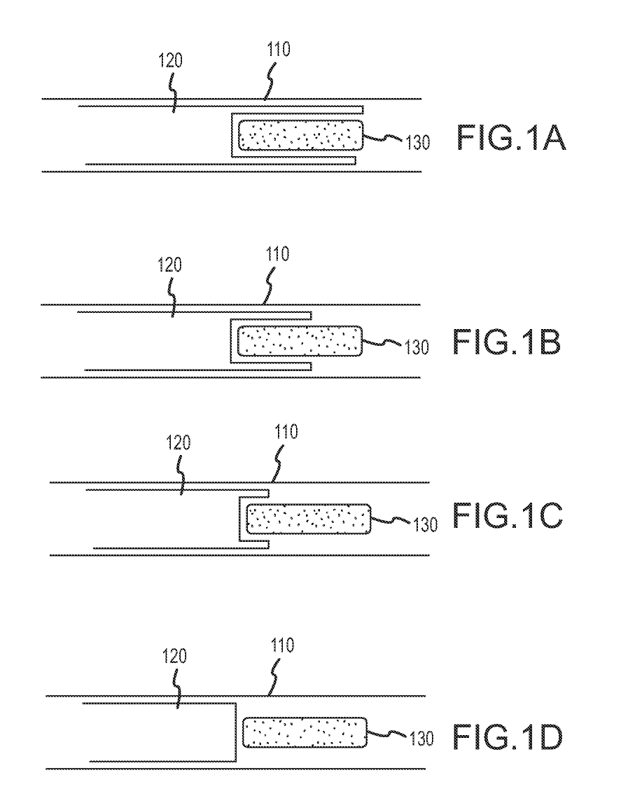

In order to place the SMPTCD without activating the SMP material, it may be necessary to use a specially designed catheter, as illustrated in FIGS. 1A-1D. As shown in FIG. 1A, the SMPTCD 130 may be fully or completely enclosed within the outer delivery catheter structure 120. This complete constraint of the SMPTCD 130 within the outer delivery catheter 120 may help to prevent premature activation of the SMPTCD 130 during placement and may also help provide better control over proper placement of the SMPTCD 130. Said another way, if the SMPTCD 130 is not completely constrained within the delivery catheter structure, as illustrated in FIGS. 1B-1D, the SMP-based material may become activated by, for example, body heat, and begin to deploy before placement of the SMPTCD 130 is complete, causing medical complications during the placement procedure.

In some embodiments, the catheter design may include the outer tubular delivery structure made from specifically selected materials which prevent premature activation of the SMP material. In other embodiments, the outer tubular delivery structure may be formed from a material having a thickness sufficient to prevent the SMP material from become activated or heated and deploying the SMPTCD before placement is completed. The material used to form the outer tubular deliver structure may be of a composition and/or thickness such that it prevents the SMP material from becoming activated or heated and deploying the SMPTCD before placement is completed.

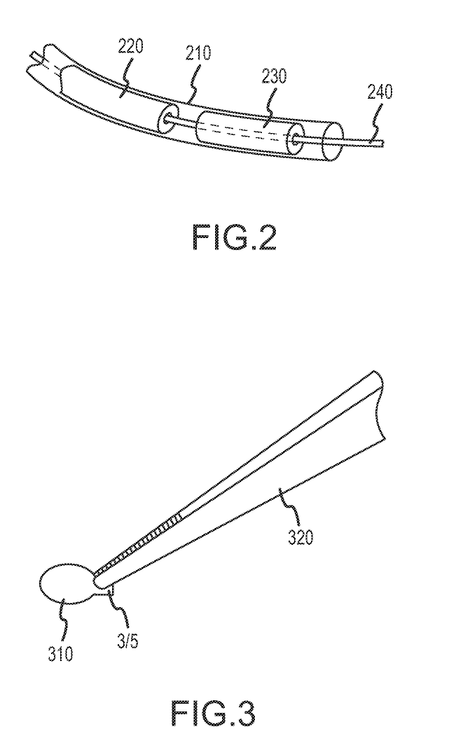

The SMP-based TCDs may also be retrieved using catheter approaches at a later date post-implantation. An exemplary catheter is shown in FIG. 2. The simplest way to remove SMP-based TCDs may be to simply grip the SMP-based TCD with a device such as a catheter and withdraw the SMP-based TCD from the body. When a catheter is used to withdraw the SMP-based TCD from the body, a catheter retrieval mechanism may grasp the SMP-based TCD and pull the SMP-based TCD into the catheter and then out of the fallopian tube and/or the body.

A retrieval catheter may include an outer tubular delivery sleeve structure 210 enclosing an inner pushing/pulling catheter structure 220. The pushing/pulling structure 220 may be used to push the SMPTCD 230 out of the outer tubular delivery sleeve structure 210 and into the fallopian tube for deployment. Of course, the pushing/pulling structure 220 may also be used to grasp and pull and SMPTCD 230 into the outer tubular sleeve structure 210 for removal or retrieval of the SMPTCD 230. As shown in FIG. 2, the pushing/pulling structure 220 may include a guidewire 240 for guiding placement of the SMPTCD 230 as well as pushing the SMPTCD 230 out of the outer tubular sleeve structure 210 and into the fallopian tube. The pushing/pulling structure may also include a retrieval mechanism, such as tweezers to grasp the SMPTCD and pull it back into the outer tubular structure for removal from the body.

Additionally, the catheter delivery and retrieval systems may be designed specifically to work with the unique working characteristics of the SMPTCDs disclosed herein. In one implementation, a size 5 French catheter was used to insert a bulb-shaped SMPTCD with good results, as described above. The catheters for use in retrieval of the SMPTCD may also have a mechanism for grasping the SMPTCD and pulling it into the catheter or through the fallopian tube for removal. The grasping mechanism may comprise any suitable means for grabbing the SMPTCD, such as pinchers, tweezers, or other similar mechanisms.

SMP-based TCDs may also be removed by several other methods including localized heating or cooling. Heating may soften the SMP-based material for easier removal. Cooling may compact the SMP-based material, such as by inactivation of the SMP-based material, for easier and less invasive (due to narrower diameter) removal. The SMP-based TCD may be either heated or cooled, and then withdrawn out of the body. In some implementations, such as after cooling if the SMPTCD is compressed, the SMP-based TCD may be drawn back into the catheter and then drawn out of the body. Heating of the SMP-based material may be accomplished by injecting sterile saline of a temperature higher than body temperature in the vicinity of the device. Similarly, cooling of the SMP-based material may be accomplished by injecting sterile saline of a temperature lower than body temperature in the vicinity of the SMPTCD. An SMP-based TCD may also incorporate heating or cooling elements within its body, which may be activated by connecting a device, such as catheter, to a heating or cooling device to generate a small electrical charge and change the temperature of the SMP-based material to soften the material or inactivate the SMP-based material. These heating or cooling elements may comprise an electrically conductive element, such as a wire or several wires. Other heating, cooling and removal techniques may be utilized herein to remove the SMP-based TCDs.

Devices utilizing SMP-based materials in a variety of SMP-based TCDs are disclosed herein, including a bulb-shaped design and a coil-shaped-design. Each of these designs will now be discussed in detail below.

### Bulb-Shaped SMPTCD Design

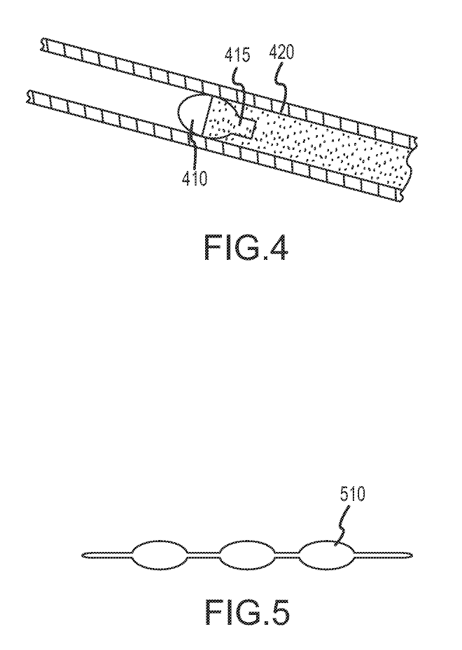

In one implementation a SMPTCD may be formed in the shape of a bulb or plug, when activated or expanded, as shown in FIGS. 3 and 4. The design of the SMPTCD may be that of a bulb-like shape meant to block the passageways or lumens of the fallopian tubes and prevent conception. The bulb-like shape may be an approximately oval-shaped device (shown in FIGS. 3 and 4) and may be referred to as a plug or bulb design herein. FIG. 3 illustrates an exemplary bulb-shaped activated device 310 held by a pair of tweezers 320. In some embodiments the bulb-shaped SMPTCD 310 may further comprise elongated end portions 315, which may make it easier to grasp the SMPTCD during removal. FIG. 4 illustrates an exemplary bulb-shaped device 410 with an elongated end 415 deployed within a clear tube 420, fully blocking the lumen and preventing the flow of a fluid 425 or other material through the tube. In some implementations, multiple bulb-shaped SMPTCDs may be used to help ensure full blockage of the fallopian tube. In other implementations a single SMPTCD may comprise one or more bulb-shaped or plug-shaped portions on one device. A SMPTCD 510 having multiple bulb-shaped portions is illustrated in FIG. 5.

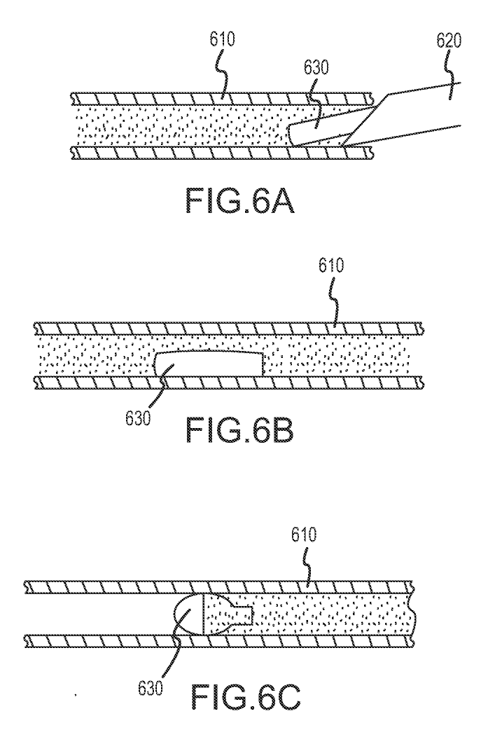

The SMPTCDs may be inserted through the cervix into one or both of the fallopian tubes. The SMPTCD may be compacted to a cylindrical shape of a small diameter and inserted into a catheter for insertion into the fallopian tubes. Once the SMPTCD is placed in a catheter, it may be inserted into the fallopian tube(s) for deployment. FIGS. 6A, 6B and 6C show a 2.4 mm diameter SMPTCD device 630 (FIG. 6C) compacted to a 1.5 mm diameter (FIG. 6B) and inserted via a size 5 French catheter 620 into a glass mould of a fallopian tube 610 (FIG. 6A). FIG. 6A illustrates the insertion of the deformed SMPTCD 630 by pushing it out of the end of a catheter 620 and into the glass mould of the fallopian tube 610. Once the SMPTCD 630 is in place, the body's natural heat activates the shape memory effect to expand the bulb-shaped device to its full 2.4 mm diameter size, as shown in FIG. 6C. Once inserted, the body's natural heat will activate the shape-memory effect of the SMP material and return the compacted cylindrical SMPTCDs to their original bulb-like shapes, effectively blocking the fallopian tubes instantaneously. Alternatively, additional heating may be applied via, for example, the injection of saline solution in the vicinity of the SMPTCD to induce faster expansion, if desired.

These SMPTCDs have a significant capacity for size change, which allows use of small delivery catheters. These SMPTCDs also have the ability to provide immediate effectiveness post-implantation. The SMPTCDs disclosed here are gentle and exhibit self-expansion, full conformance to tortuous local anatomy, and the ability to elute drugs as needed. Further, these SMPTCDs are a reversible method of sterilization because these devices can be retrieved at a later date.

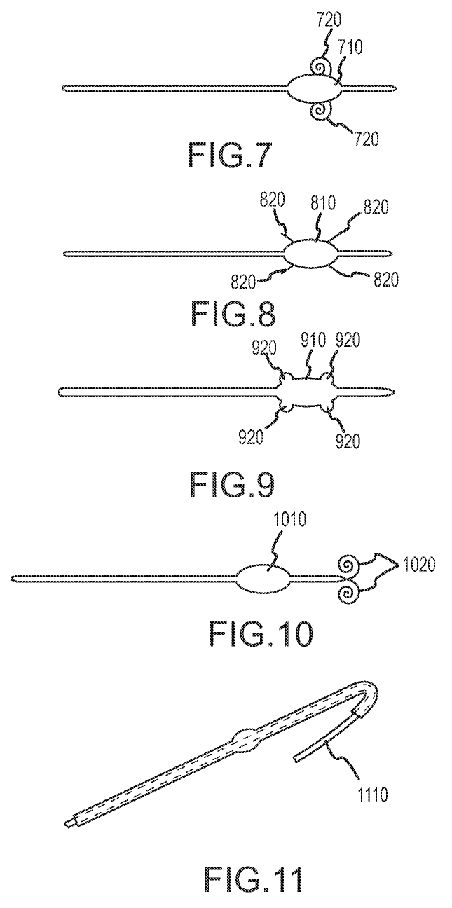

Several additional adjustments may be made to the design of the SMPTCDs to enhance their functionality. In some implementations, barbs, hooks, anchors, or other protrusions may be added to the SMPTCDs to help secure or anchor the SMPTCD within the lumen or walls of the fallopian tube. In some embodiments the barbs, hooks or anchors may be positioned on the bulb-shaped body of the SMPTCDs, as shown in FIGS. 7, 8, and 9. FIG. 7 illustrates an exemplary embodiment of a SMPTCD 710 having hooks 720 positioned on the body portion of the SMPTCD 710. FIG. 8 illustrates an exemplary embodiment of a SMPTCD 810 having barbs 820 positioned on the body portion of the SMPTCD 810. FIG. 9 illustrates an exemplary embodiment of a SMPTCD 910 having anchors 920 positioned on the main body portion of the SMPTCD 910.

In other embodiments, the hooks or anchors may be positioned on an elongated end portion of the device, away from the bulb-shaped body portion, as shown in FIG. 10. FIG. 10 illustrates an exemplary embodiment of a SMPTCD 1010 having hooks 1020 positioned on an elongated end portion of the SMPTCD 1010. In this embodiment the hook or anchor may be hooked onto or anchored on an end of a fallopian tube (on the ovarian end or the uterine end) to hold the SMPTCD in place. The distance between the hook and the bulb-shaped body portion can vary over a wide range, such as from 0.1 mm to 40 mm. The hook or anchor may be positioned on either end of the SMPTCD, such as on the proximal end (uterine end) or on the distal end (ovarian end). These implementations may help prevent or minimize any movement of the SMPTCD within the tube, enhancing the secure fit within the fallopian tube and increasing the effectiveness of the contraceptive or sterilization.

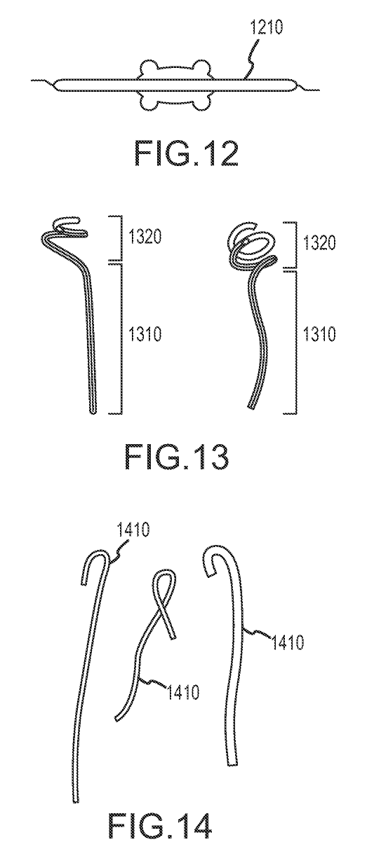

In other implementations, a thin string or wire may be added or attached to the SMPTCD, for example, by embedding it within the device during polymerization. Examples of the addition of a string or wire are shown in FIGS. 11 and 12. FIG. 11 illustrates a SMPTCD that includes a guidewire 1110. FIG. 12 Illustrates a SMPTCD that includes a central shaft portion 1210. The guidewire 1110 or other central shaft portion 1210 may be used to help control or guide the placement of the SMPTCD within a patient. The guidewire 1110 or central shaft portion 1210 may also be used for pushing the SMPTCD out of a catheter during delivery and/or for grasping the SMPTCD and pulling it into the catheter during retrieval. Once the SMPTCD is in proper position within the patient, the guidewire 1110 or other central shaft portion 1210 may be removed. Alternatively, in some embodiments the guidewire 1110 or other central shaft portion 1210 may be permanent and remain within the SMPTCD. The guidewire 1110 or other central shaft portion 1210 may be formed of fabric, metal, polymer, or other materials, and may extend through the length of the SMPTCD. For example, the guidewire or other central shaft portion may be manufactured from a metal material to enhance radio-opacity of the SMPTCD and provide a pathway to conduct electricity for heating or cooling of the polymer to active the SMP material. The addition of the guidewire or other central shaft portion may provide additional structural support to the SMPTCD, and may also increase the tensile strength of the SMPTCD.

### Coil-Shaped SMPTCD Design

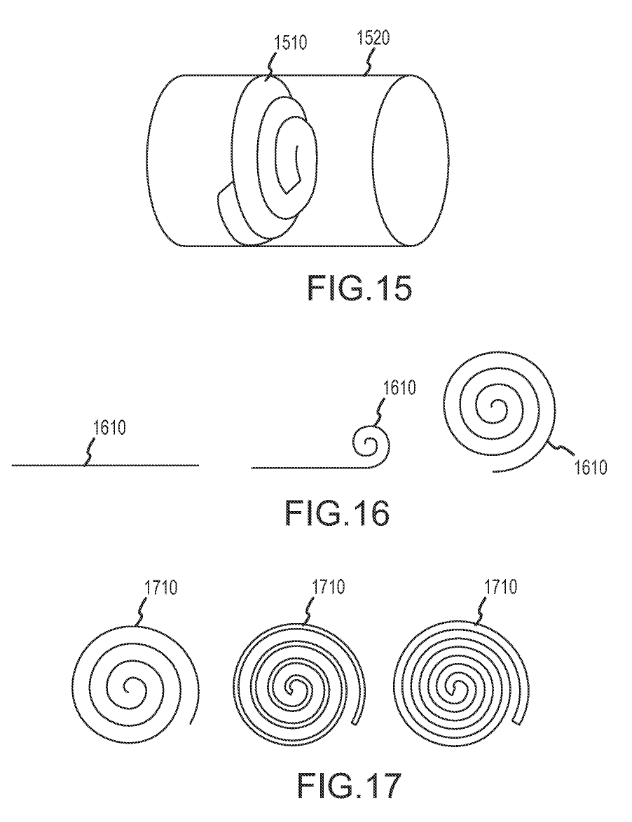

A SMPTCD having a coil-shaped design is also disclosed and shown in FIGS. 13 and 14. The coil-shaped SMPTCD may be compacted into an elongated or linear strip of material, as shown in FIG. 14, and may form a coil-shape upon activation, as shown in FIG. 15. FIG. 15 illustrates a coil-shaped SMPTCD 1510 fully deployed and blocking a clear tube 1520. FIG. 13 illustrates a partially activated coil-shaped SMPTCD, showing the linear compacted portion 1310 at the bottom and the coil-shaped portion 1320 at the top of the SMPTCD.

Advantages of a coil-type design include the use of a small delivery catheter and gentle self-expansion of the device. Before insertion, the SMP-based TCD coil is uncoiled to a straight shape 1410 that fits easily into a catheter, which may then be inserted into the fallopian tubes. Once inserted into the fallopian tube and free of the catheter, the body's natural heat will activate the shape-memory effect and return the SMPTCD to its original coiled shape 1510, effectively occluding the fallopian tube, as shown in FIG. 15.

The combination of SMP-based material with the 2-HEMA polymer as described above provides unique structure and function that may provide significant improvements over other materials used in TCDs. For example, the SMP-based material may provide an ability to expand the SMPTCD 1510 significantly (as shown in FIG. 15) from a compacted state in the delivery catheter, and thereby allow for immediate effectiveness as a contraceptive/sterilization product. The use of the 2-HEMA polymer provides a water absorption aspect to allow further expansion over time to “lock” the SMPTCD 1510 in place to ensure the SMPTCD 1510 will be permanently implanted. The ability to swell by water absorption also provides the ability to fine-tune how well the SMPTCD 1510 conforms to the tube 1520 in which it is placed, which may be particularly important for complex anatomy.

FIG. 16 illustrates the gradual coiling activation of the SMPTCD 1610, shown from left to right. FIG. 16 illustrates a compacted SMPTCD 1610 (at left) and the gradual coiling of the SMPTCD 1610 (at right). Coil shapes have been manufactured to be compacted into catheters with internal diameters ranging from 0.9 to 2.0 mm, and expand into tubes of diameters ranging from 1.5 to 4.0 mm.

The coil-shaped SMPTCDs may require some fibrous growth to completely block the fallopian tube. Thus, the coil-shaped SMPTCDs may also incorporate fibrous structures or mesh in areas to encourage additional fibrous growth over time. Because the coil-shaped SMPTCD may have delayed sterilization effectiveness, it may be desirable to use the coil-shaped SMPTCD in combination with the bulb-shaped SMPTCD, to provide immediate effectiveness. In some situations, the coil-shaped SMPTCD may be used for permanent sterilization if it accumulates fibrous growth over time.

In other embodiments, coil-shaped SMPTCDs may incorporate barbs, hooks or anchors, and/or may be used in combination with bulb-shaped SMPTCDs and/or stents as described above. The coiled SMPTCDs may also incorporate medications and/or be co-polymerized with thin strings, for example, made of fabric, metal, or other polymers, to increase tensile strength, also as described above.

The coil-shaped SMPTCDs may be fabricated from the same or similar materials described above with reference to the bulb- or plug-shaped SMPTCD and/or they may also incorporate or substantially comprise a hydrogel, causing the polymer material to soften and swell as it absorbs water over time. It may be desirable to use a hydrogel, such as 2-hydroxyethyl methacrylate (2-HEMA) in place of, or in conjunction with, the tert-butyl acrylate. FIG. 17 illustrates the swelling of a coil-shaped SMPTCD 1710 incorporating a hydrogel. FIG. 17 illustrates a coil-shaped SMPTCD 1710 before swelling (at left) and then shows the gradual swelling of the hydrogel material (at right) over time. The incorporation of the hydrogel material increases the thickness of the coil-shaped SMPTCD 1710, helping to more completely block the fallopian tube to achieve more effective sterilization.

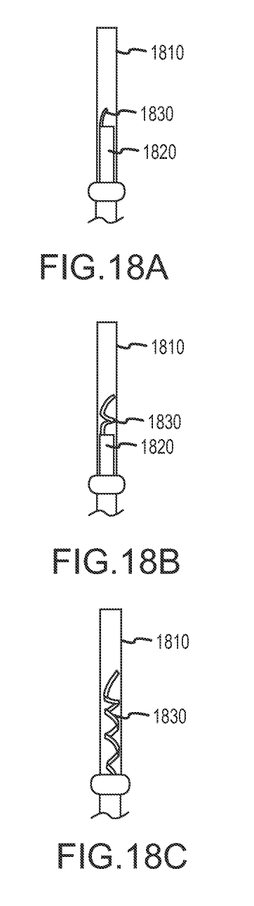

Once the SMPTCD is placed in a catheter, it may be delivered into the fallopian tubes for deployment. FIGS. 18A, 18B and 18C show a 0.9 mm diameter straightened coil 1830 deployed via a 5 French catheter 1820 into a glass tube 1810 in pulsatile flow maintained at body temperature. FIG. 18A illustrates the SMPTCD 1830 packed within the catheter 1820 and being pushed out of the catheter 1820. FIG. 18B illustrates more of the coiled SMPTCD 1830 as it continues to be pushed out of the catheter 1820 and into the glass tube 1810. Once the SMPTCD 1830 is delivered by being pushed out of the catheter 1820, the SMPTCD 1830 begins to coil, as shown in FIG. 18C, invoking the shape memory effect.

In addition to the above described materials, both the coil- and bulb-shaped SMPTCDs may be manufactured using a combination of SMP and non-SMP materials. The addition of the non-SMP materials may help to increase mechanical strength of the device. In one example, different weight fractions of reinforcing fibers (non-SMP materials) may be added to enhance durability and resistance to tearing. This mixed polymer may be formed by selecting the appropriate glass transition temperature and appropriate percentage of crosslinking monomer and then blending in the typical fashion. After blending, an appropriate amount of photoactivated initiator may be added. The mixture may be agitated until the initiator is completely dissolved. Once the initiator has been dissolved the mixture is ready for polymerization and may be set aside. An appropriately shaped mould may be made, typically out of glass slides held 1-2 mm apart by a non-reactive rubber spacer. Once the mould is prepared, the reinforcing fibers (i.e. non-SMP materials) may be added to the mold, and it is sealed closed. The reinforcing fibers used in this process may be short, for example averaging 150μ in length and 7-10μ in diameter. (However, it is contemplated that the length of the fibers may be altered.) The prepared monomer solution may then be injected into the mould. The entire mould may then be vigorously agitated both before and during polymerization to ensure the reinforcing fibers will be evenly distributed in the final product.

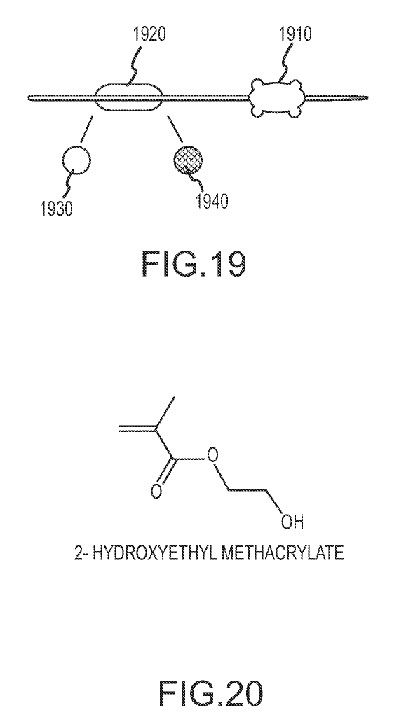

In additional implementations, either the coil-shaped or the bulb-shaped SMPTCDs 1910 may be used in conjunction with an SMP stent 1920 as shown in FIG. 19. The stent 1920 may be formed of the SMP material, as described above, and may be crushed or compacted into a more linear shape for delivery as described with reference to the SMPTCDs herein. The stent 1920 may be hollow with solid walls 1930 or may comprise a mesh matrix of fibers 1940, as shown in FIG. 19. The presence of the stent 1920 may encourage post-implant fibrous growth which will provide permanent blockage of the fallopian tube. Because the post-implant fibrous growth takes time to grow, the use of the stent 1920 will have delayed sterilization effectiveness. In some situations, it may be desirable to implant a bulb-shaped SMPTCD 1910, to provide immediately effective sterilization, and a stent SMPTCD 1920, to provide delayed but permanent sterilization.

The drawings attached hereto are intended to further illustrate and exemplify the SMPTCDs described herein. These exemplary drawings are for purposes of illustration only and the dimensions, sizes and shapes reflected in the drawings attached hereto may vary. These SMPTCDs may be formed in a variety of sizes and shapes and exact measurements given above are exemplary in nature only and are not meant to be limiting.

The above description, examples and data provide a complete description of the structure and use of example embodiments of the invention. Although various embodiments of the invention have been described above with a certain degree of particularity, or with reference to one or more individual embodiments, those skilled in the art could make numerous alterations to the disclosed embodiments without departing from the spirit or scope of this invention. It is intended that all matter contained in the above description and shown in the accompanying drawings shall be interpreted as illustrative only of particular embodiments and not limiting. Changes in detail or structure may be made without departing from the basic elements of the invention.

What is claimed is:

1. A transcervical contraceptive device comprising a shape memory polymer material adapted to block a fallopian tube upon activation.

2. The device of claim 1, wherein the device forms a bulb-shape upon activation.

3. The device of claim 1, wherein the device forms a coil-shape upon activation.

4. The device of claim 1, wherein the device forms a coil-shape and a bulb-shape upon activation.

5. The device of claim 1, wherein the device further comprises a hydrogel material causing the device to swell upon activation.

6. The device of claim 5, wherein the hydrogel material comprises 2-hydroxyethyl methacrylate (2-HEMA).

7. The device of claim 1, further comprising an exposed fibrous mesh cross-section embedded within the shape memory polymer material for stimulating additional fibrous growth through the device.

8. The device of claim 1 further comprising an exposed fiber embedded within the device.

9. The device of claim 8, wherein the fiber is electrically conductive.

10. The device of claim 8, wherein energizing the electrically conductive fiber changes a temperature of the device and thereby activates the device.

11. The device of claim 1 wherein the device forms an anchoring protrusion upon activation for securing the device within a fallopian tube.

12. The device of claim 1, wherein the device is adapted to expand from an initially contracted shape upon exposure to a stimulus.

13. The device of claim 12, wherein the stimulus is heat or light.

14. The device of claim 1, further comprising a medication embedded within the device.

15. The device of claim 1, further comprising a medication coated onto the device.

16. (canceled)

17. (canceled)

18. (canceled)

19. (canceled)

20. (canceled)

21. The device of claim 1 further configured for delivery within a human body via a tubular lumen.

22. The device of claim 1 further comprising a shaft extending from one or more ends of an occluding structure of the device.

23. The device of claim 22, wherein the shaft further comprises an anchoring structure.

24. The devise of claim 12, wherein the stimulus is body heat of a patient.

25. A method of manufacturing a transcervical sterilization device comprising
forming a shape memory polymer material into a first shape configured to block a fallopian tube;
changing an environmental temperature to a deformation temperature of the shape memory polymer material; and
deforming the first shape into a second shape configured for delivery via catheter into a fallopian tube.

26. The method of claim 25 further comprising changing the environmental temperature after forming the second shape to a storage temperature below a glass transition temperature of the shape memory polymer material.

27. The method of claim 25 further comprising forming the first shape as a bulbous shape.

28. The method of claim 25 further comprising forming the first shape as a coil shape.

29. The method of claim 25 further comprising forming the first shape to include an anchoring protrusion.

30. The method of claim 25 further comprising embedding an exposed fibrous mesh within the shape memory polymer material.

31. The method of claim 25 further comprising embedding an electrically conductive wire within the shape memory polymer material.

32. The method of claim 25 further comprising incorporating a hydrogel material within the shape memory polymer material to cause the device to swell upon activation.

33. The method of claim 25 further comprising incorporating a dissolving material within a polymer network of the device.

34. The method of claim 33, wherein the dissolving material is a medication.

35. The method of claim 25 further comprising coating the device with a dissolving material.

36. The method of claim 35, wherein the dissolving material is a medication.

37. The method of claim 25 further comprising selecting the shape memory polymer material to have a glass transition temperature substantially the same as a normal temperature of a human or animal patient to receive the device.

38. The method of claim 25 further comprising selecting the deformation temperature to be above a glass transition temperature of the shape memory polymer material.
